(19) United States
(12) Patent Application Publication
(10) Pub. No.: US 2025/0297203 A1
(43) Pub. Date: Sep. 25, 2025

(54) CELL DISPENSING DEVICE

(71) Applicant: Funai Electric Co., Ltd., Osaka (JP)

(72) Inventors: Bruce D. Gibson, Lexington, KY (US); David A. MADILL, Lexington, KY (US)

(73) Assignee: Funai Electric Co., Ltd., Osaka (JP)

(21) Appl. No.: 18/609,086

(22) Filed: Mar. 19, 2024

(51) Int. Cl. C12M 1/26; C12M 1/00

(52) U.S. Cl. CPC C12M 33/12; C12M 29/06

(57) ABSTRACT

A cell dispensing device and method of capturing and dispensing individual cells to an analytical substrate. The device includes a thermal fluid ejection head having a plurality of cell ejection chambers formed in an aspiration channel layer attached to a first semiconductor substrate. A cell ejector is disposed on the first semiconductor substrate in each of the cell ejection chambers, and aspiration channels are formed in the aspiration channel layer in flow communication with at least some of the cell ejection chambers. A cell ejection nozzle layer contains cell ejection ports therein, wherein the cell ejection nozzle layer is attached to the aspiration channel layer. An activation circuit for each cell ejector is provided in the plurality of cell ejection chambers. An aspiration device is provided in fluid flow communication with at least some of the cell ejection chambers through the aspiration channels.

### TECHNICAL FIELD

The disclosure is directed to dispensing cells into devices for analytical purposes and in particular to devices and methods for capturing individual cells and dispensing the individual cells on demand into an analytical device.

### BACKGROUND AND SUMMARY

In the medical field, in particular, there is a need for automated sample preparation and analysis. The analysis may be colorimetric analysis or require the staining of samples to better observe the samples under a microscope. Such analysis may include drug sample analysis, blood sample analysis and the like. In the analysis of blood, for example, blood is analyzed to provide a number of different factors that are used to determine the health of an individual. When there are a large number of patients that require blood sample analysis, the procedures may be extremely time consuming. Also, there is a need for accurate preparation of the samples so that the results can be relied on. For example, the ability to dispense one cell at a time to an analytical device is very useful for a variety of life science applications where cells are tested for reactions to drugs and other stimuli. Conventional cell picking machines with cameras that are capable of picking a single cell at a time and placing the cell in a well of a micro-well plate are extremely expensive.

In view of the foregoing, what is needed is an apparatus that is configured to isolate individual cells and a minor amount of fluid from a fluid containing a plurality of cells and to dispense one of the individual cells to a predetermined receptacle in a micro-well plate using a thermal cell ejector.

In view of the foregoing, an embodiment of the disclosure provides a cell dispensing device and method of capturing and dispensing individual cells to an analytical substrate. The device includes a thermal fluid ejection head having a plurality of cell ejection chambers formed in an aspiration channel layer attached to a first semiconductor substrate. A thermal cell ejector is disposed on the first semiconductor substrate in each of the cell ejection chambers, and aspiration channels are formed in the aspiration channel layer in flow communication with at least some of the cell ejection chambers. A cell ejection nozzle layer contains cell ejection ports therein, wherein the cell ejection nozzle layer is attached to the aspiration channel layer. An activation circuit is provided for each thermal cell ejector in the plurality of cell ejection chambers. An aspiration device is provided in fluid flow communication with at least some of the cell ejection chambers through the aspiration channels.

In another embodiment, there is provided a method of capturing and dispensing individual cells to an analytical substrate. The method includes providing a cell dispensing device that includes a thermal fluid ejection head having a plurality of cell ejection chambers formed in an aspiration channel layer attached to a first semiconductor substrate. A thermal cell ejector is disposed on the first semiconductor substrate in each of the cell ejection chambers. Aspiration channels are formed in the aspiration channel layer in flow communication with at least some of the cell ejection chambers. A cell ejection nozzle layer containing cell ejection ports therein is attached to the aspiration channel layer. An activation circuit is provided for each thermal cell ejector in the plurality of cell ejection chambers. An aspiration device is provided in fluid flow communication with at least some of the cell ejection chambers through an aspiration port in fluid flow communication with the aspiration channels. A fluid containing cells is applied to the cell ejection nozzle layer. A negative pressure is applied to the aspiration port to pull cells in the fluid from the cell ejection nozzle layer through the cell ejection ports into the cell ejection chambers. Excess fluid containing cells is removed from the cell ejection nozzle layer. A determination is made as to which cell ejection chambers have cells therein. The thermal cell ejectors for the cell ejection chambers having cells therein are activated to deposit the cells onto the analytical substrate.

In another embodiment, there is provided a method of capturing and dispensing individual cells to an analytical substrate. The method includes providing a cell dispensing device containing a thermal fluid ejection head attached to a fluid reservoir. The thermal fluid ejection head has a plurality of cell ejection chambers formed in an aspiration channel layer attached to a first semiconductor substrate. A thermal cell ejector is disposed on the first semiconductor substrate in each of the cell ejection chambers. Aspiration channels are formed in the aspiration channel layer in flow communication with at least some of the cell ejection chambers. A cell ejection nozzle layer containing cell ejection ports therein is attached to the aspiration channel layer. An activation circuit is provided for each thermal cell ejector in the plurality of cell ejection chambers. An aspiration device is provided in fluid flow communication with at least some of the cell ejection chambers through an aspiration port in fluid flow communication with the aspiration channels. A negative pressure is applied to the aspiration port to pull a fluid containing cells from the fluid reservoir into the cell ejection chambers. Excess fluid containing cells is removed from the cell ejection chambers. A determination is made to determine which cell ejection chambers have cells therein, and the thermal cell ejectors for the cell ejection chambers having cells therein are activated to deposit the cells onto the analytical substrate.

In some embodiments, the thermal fluid ejection head is attached to a fluid reservoir.

In some embodiments, the thermal fluid ejection head further includes a separate fluid ejection structure containing a second semiconductor substrate having a plurality of fluid ejectors thereon and a fluid supply via etched therethrough. A flow feature layer is attached to the second semiconductor substrate, and a nozzle plate is attached to the flow feature layer. The fluid supply via is in fluid flow communication with a fluid in the fluid reservoir. The separate fluid ejection structure is devoid of the aspiration channels in the flow feature layer.

In some embodiments, a barrier wall is provided on the cell ejection nozzle layer circumscribing the cell ejection ports therein.

In some embodiments, the barrier wall has a height ranging from about 50 microns to about 4 millimeters.

In some embodiments, a fluid supply via is etched through the first semiconductor substrate in fluid flow communication with the fluid reservoir, wherein the fluid supply via is configured for providing fluid through fluid channels to the cell ejection chambers.

In some embodiments, the cell ejection ports in the cell ejection nozzle layer have a diameter ranging from about 10 to about 50 microns.

In some embodiments, the cell ejection chambers have a width and length ranging from about 10 to about 60 microns and a depth ranging from about 15 to about 30 microns.

An advantage of the disclosed embodiments is that the disclosed device enables an ability to dispense a single cell at a time onto an analytical substrate much more inexpensively than with the use of conventional cell dispensing equipment. The disclosed method and apparatus avoids the use of complicated cell picking machines that are used to insert one cell at a time into a standard well of a micro-well plate.

### BRIEF DESCRIPTION OF THE DRAWINGS

FIGS. 1-2 are cross-sectional schematic views, not to scale, of an ejection head configured for capturing a single cell according to an embodiment of the disclosure.

FIG. 3 is an exploded view, not to scale, of a portion of the ejection head of FIG. 1.

FIG. 4 is a plan view, not to scale, of a series of aspiration channels and an aspiration port for the ejection head of FIG. 1 according to one embodiment of the disclosure.

FIG. 5 is a perspective view, not to scale, of a series of aspiration channels and an aspiration port for the ejection head of FIG. 1 according to another embodiment of the disclosure.

FIG. 6 is a perspective view, not to scale, of a cartridge containing an ejection head according to an embodiment of the disclosure.

FIG. 7 is an electrical schematic view of a portion of an activation circuit for an ejection head according to the disclosure.

FIG. 8 is an enlarged perspective view, not to scale, of a portion of the ejection head on the cartridge of FIG. 6.

FIG. 9 is a perspective view, not to scale, of a cartridge containing an ejection head according to another embodiment of the disclosure.

FIG. 10 is a plan view, not to scale, of a conventional fluid ejection head for cartridge of FIG. 9.

FIG. 11 is a plan schematic view, not to scale, of an ejection head according to another embodiment of the disclosure.

FIG. 12 is a cross-sectional view, not to scale, of a portion of the ejection head of FIG. 10.

### DETAILED DESCRIPTION OF EXEMPLARY EMBODIMENTS

As used herein “cell” refers to a single biological cell that may include a cell from a plant, animal, bacteria, virus, and the like.

As used herein “analytical substrate” means any substrate used for analytical purposes such as a well plate, glass slide, bacterial growth media, and the like.

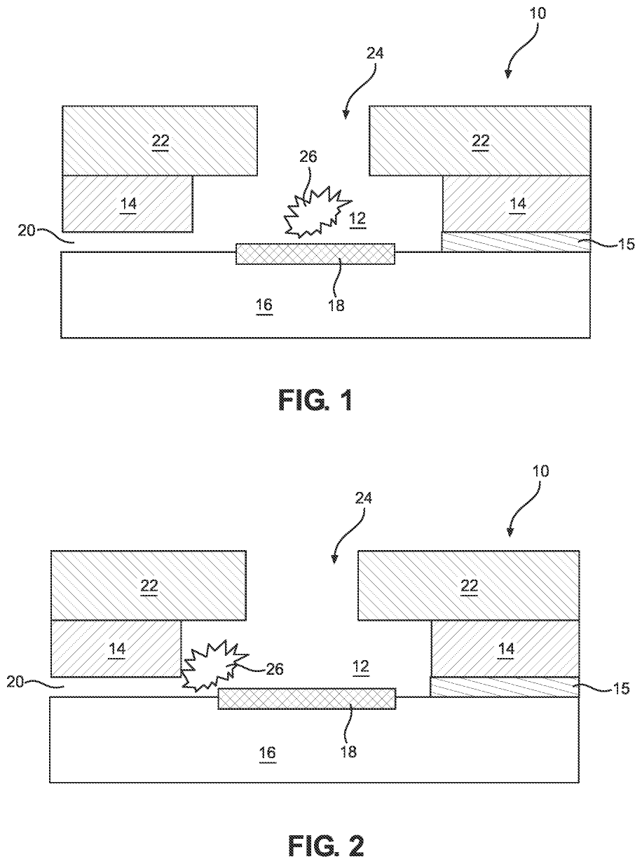

The apparatus for isolating and dispensing a single cell is based generally on the use of thermal cell ejectors to heat a cell containing fluid so that the cell and fluid are ejected from the apparatus onto an analytical substrate for testing and analysis. With reference to FIGS. 1 and 2, the apparatus includes a thermal fluid ejection head 10 having a plurality of cell ejection chambers 12 formed in an ejection chamber layer 14 and an aspiration channel layer 15. The aspiration channel layer 15 is attached to a first semiconductor substrate 16. A thermal cell ejector 18 is disposed on the first semiconductor substrate 16 in each of the cell ejection chambers 12. The aspiration channel layer 15 also has an aspiration channel 20 formed therein in flow communication with at least some of the cell ejection chambers 12. A cell ejection nozzle layer 22 containing cell ejection ports 24 therein is attached to the ejection chamber layer 14. Each of the layers 14, 15 and 22 may be formed separately and laminated to one another and the combined layers 14, 15 and 22 attached to the semiconductor substrate 16.

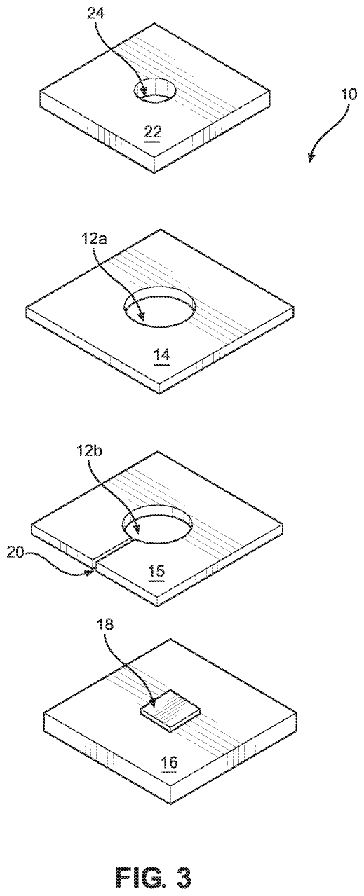

The thermal cell ejector 18 may be formed by conventional thin film technology used to form a heater stack on the first semiconductor substrate 16. The cell ejection chamber 12 is sized to collect a single cell 26 along with a minor amount of fluid that can be heated to expel the cell 26 through the cell ejection port 24. Accordingly, the cell ejection chamber 12 may have a depth provided by the ejection chamber layer 14 and aspiration channel layer 15 ranging from about 15 to about 30 microns and a width and length ranging from about 10 to about 60 microns. The cell ejection port 24 may have a diameter ranging from about 10 to about 50 microns. Accordingly, the cells 26 may have a size ranging from about 10 to about 60 microns. The aspiration channel 20 formed in the aspiration channel layer 14 is sized based on the size of the cell 26 to enable the cell 26 to block the aspiration channel 20 thereby reducing the fluid flow to the cell ejection chamber 12. The meniscus of the cell ejection port 24 holds the cell 26 and a minor amount of fluid (not shown) in the cell ejection chamber 12. FIG. 3 is an exploded view, not to scale, of the portion of the ejection head of FIG. 1 showing the two portions of the cell ejection chamber 12a and 12b provided by layers 14 and 15.

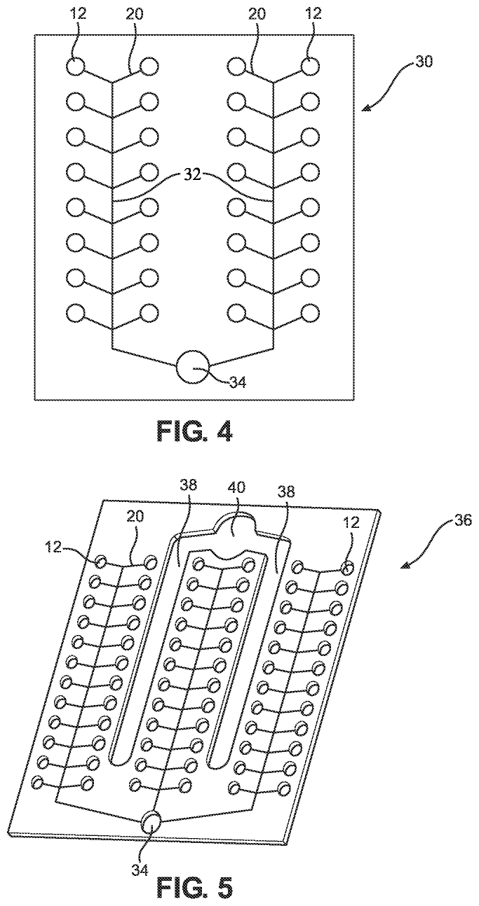

FIG. 4 is a plan view of an aspiration channel layer 30 according to one embodiment of the disclosure containing a plurality of cell ejection chambers 12 and a plurality of aspiration channels 20 connected to the plurality of cell ejection chambers 12. The plurality of aspirations channels 20 are connected to common aspiration channels 32 that leads to an aspiration port 34 that is in flow communication with an aspiration device (not shown). When a negative pressure is applied to the aspiration channels 32 from the aspiration port 34, fluid containing cells are caused to flow through the cell ejection ports 24 from the cell ejection nozzle layer 22 and into the cell ejection chambers 12.

FIG. 5 is a perspective view of an aspiration channel layer 36 according to another embodiment of the disclosure. Like the embodiment shown in FIG. 4, aspiration channels 20 are connected to cell ejection chambers 12 and the aspiration channels 20 are in flow communication with the aspiration port 34. However, unlike the embodiment of FIG. 4, the aspiration channel layer 36 also includes fluid flushing channels 38 in flow communication with a fluid flushing port 40. By applying a negative pressure to the fluid flushing port 40, excess fluid on the surface of the cell ejection nozzle layer 22 is caused to flow into the flushing channels 38 and away from the cell ejection ports 24 to prevent more than one cell from entering each cell ejection chamber 12.

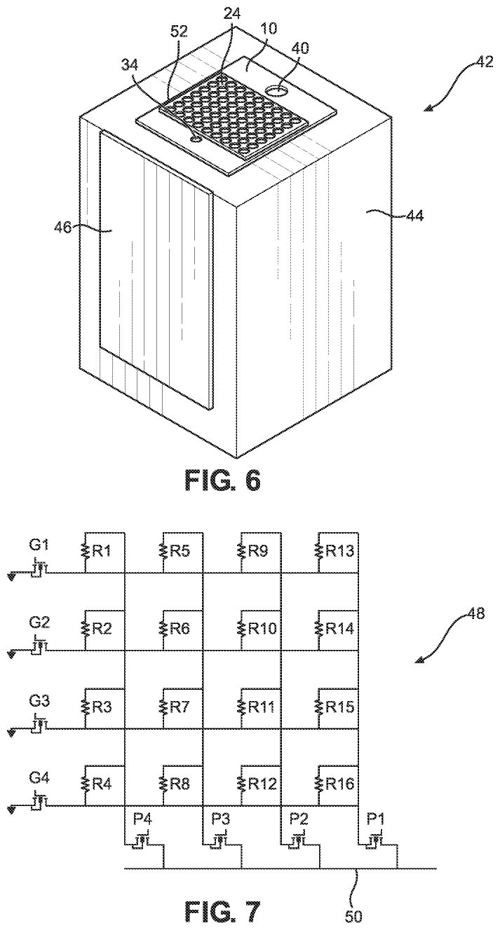

As shown in FIG. 6, the thermal fluid ejection head 10 containing a plurality of cell ejection ports 24 is attached to a cartridge 42 that has a body 44 that enables the cartridge 42 to be attached to a fluid ejection device for control of the cell ejectors 18. The cell ejection ports 24 are tightly packed on the thermal fluid ejection head 10. The thermal fluid ejection head 10 also contains the aspiration channels 20 and fluid flushing channels 38 described above which are not shown in FIG. 6. A flexible circuit 46 is attached to the body 44 of the cartridge 42 for controlling activation of the thermal cell ejectors 18 in the cell ejection chambers 12 associated with the cell ejection ports 24. In some embodiments, the body 44 of the cartridge 42 does not contain fluid. In other embodiments, described below, the body 44 of the cartridge 42 may contain fluid. Activation of thermal cell ejectors 18 for the tightly packed cell ejection ports 24 may be provided by an activation circuit that provides for activation of individual thermal cell ejectors 18 in the rows and columns for the tightly packed cell ejection ports 24 shown in FIG. 6. A portion of a representative activation circuit 48 is illustrated in FIG. 7 wherein P1-P4 represent power devices that can be activated to provide power from power input 50 to one or more thermal cell ejectors represented by R1-R16. G1-G4 represent ground devices that can be activated for one or more of thermal cell ejectors R1-R16. Accordingly, an individual thermal cell ejector, such as thermal cell ejector R6 may be activated by activating device P3 and device G2.

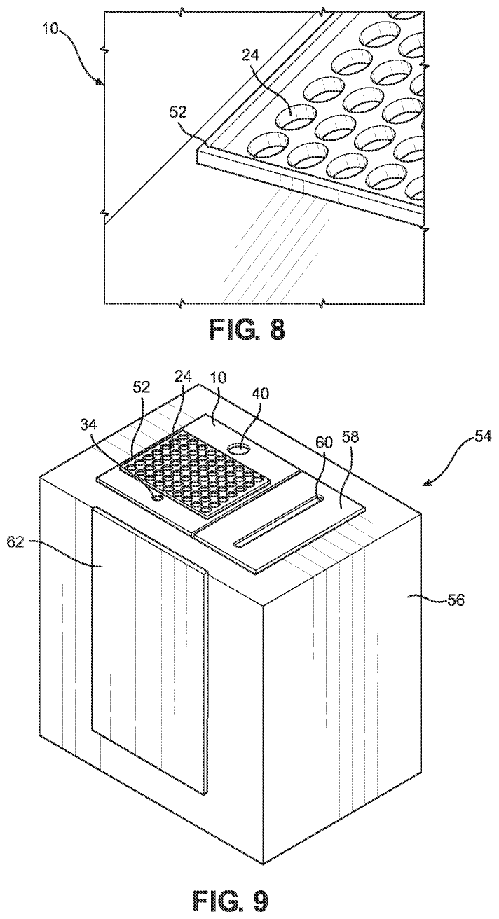

Cells may be inserted into the individual cell ejection ports 24 by flooding the thermal fluid ejection head 10 with a fluid containing the cells. In order to enable the cells to enter the cell ejection chambers 12 through cell ejection ports 24, a raised barrier wall 52 may be provided around the tightly packed cell ejection ports 24 as shown in FIG. 8 so that the aspiration port 34 and flushing port are outside of the raised barrier wall 52. The raised barrier wall 52 may be made using a layered microelectromechanical systems (MEMS) process to provide the raised barrier wall 52 having a height of about 50 microns. In an alternative process, the raised barrier wall 52 may be made of a plastic wall attached to the thermal fluid ejection head using an adhesive such as a die bond adhesive or an encapsulation adhesive. The plastic wall may have a height of about 2 to about 4 millimeters.

In order to use the devices illustrated in FIGS. 4-8, the cartridge 42 containing the thermal fluid ejection head 10 is oriented with the thermal fluid ejection head 10 facing upward. A pipette is used to apply a fluid containing cells to the surface of the thermal fluid ejection head 10 so that at least some of the fluid and cells are able to flow through the cell ejection ports 24 into cell ejection chambers 12. A negative pressure is applied to the aspiration port 34 to assist the fluid and cells to flow through the cell ejection ports 24 into the cell ejection chambers 12. The amount of negative pressure applied to the aspiration port 34 should be less than a meniscus force that holds the fluid in the cell ejection chamber. A negative pressure is also applied to the fluid flushing port 40 to remove excess fluid from the surface of the thermal fluid ejection head 10. An optical method such as a microscope or other optical inspection device may be used to determine which cell ejection chambers 12 contain a cell. The thermal fluid ejection head 10 is inverted and the thermal cell ejectors 18 for the cell ejection chambers containing a cell may be activated to eject the cell from the cell ejection chambers 12 containing a cell onto an analytical substrate.

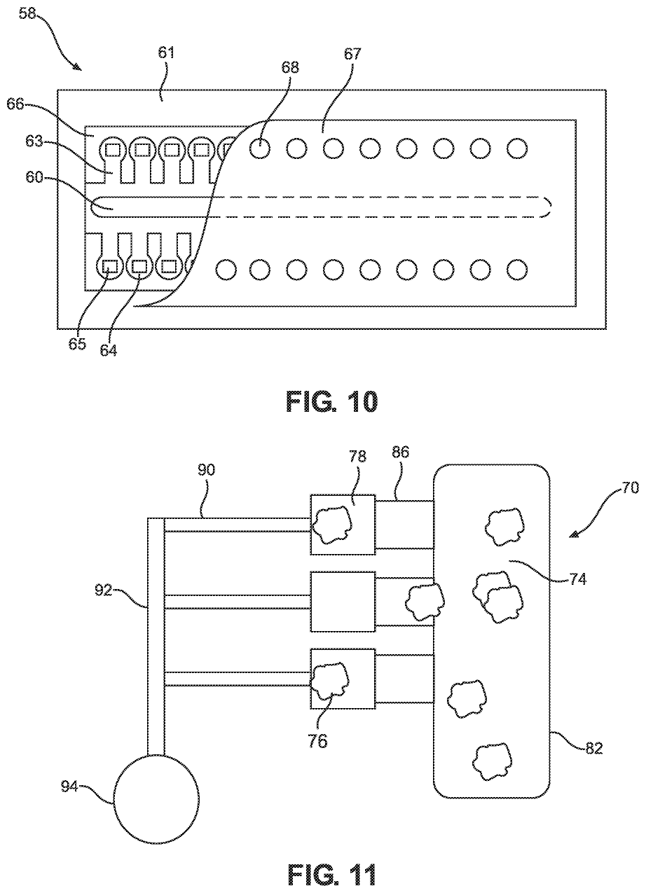

In another embodiment, illustrated in FIGS. 9 and 10 a cartridge 54 having a body 56 may include a conventional thermal fluid ejection head 58 containing a fluid supply via 60 etched through a second semiconductor substrate 61 to provide fluid from the body 56 of the cartridge 54 through fluid channels 63 to fluid chambers 64 containing fluid ejectors 65 adjacent to the fluid supply via 60. The fluid channels 63 and fluid chambers 64 are formed in a flow feature layer 66 that is attached to the second semiconductor substrate 61. A nozzle plate 67 containing nozzle holes 68 is attached to the flow feature layer 66. Accordingly, the body 56 of the cartridge 54 may be filled with a buffer fluid or other fluid for use with the thermal fluid ejection head 10 to provide the buffer fluid or other fluid to the analytical substrate along with the cells ejected from the thermal fluid ejection head 10. The body 56 of the cartridge 54 may be prefilled with the buffer fluid or other fluid or a fluid may be pipetted into the body 56 of the cartridge 54 before use. The cartridge 54 also contains a flexible circuit 62 for controlling activation of the thermal cell ejectors 18 on the thermal fluid ejection head 10 and the fluid ejectors on the fluid ejection head 58. In order to eject a cell from the cartridge 54 of FIG. 9, the same procedure may be used as is used with the cartridge of FIG. 6.

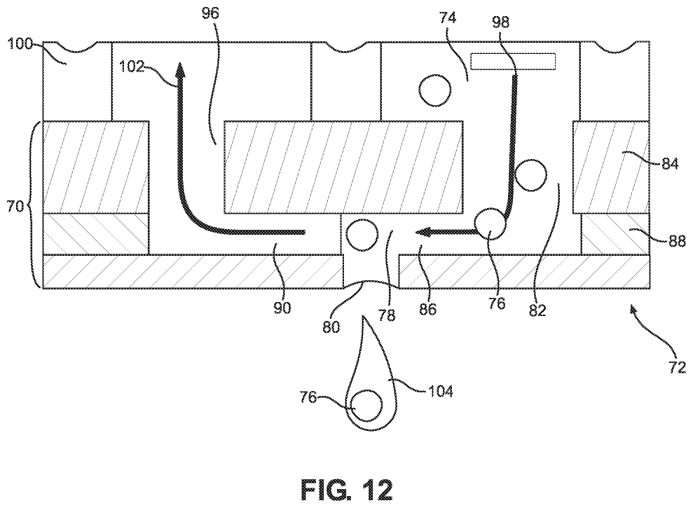

Another embodiment of the disclosure is illustrated schematically in FIGS. 11 and 12. FIG. 11 is a plan view of a portion of an ejection head 70 attached to a fluid cartridge 72 that contains a fluid 74 containing cells 76 to be dispensed to an analytical substrate. FIG. 12 is a cross-sectional view of the fluid cartridge 72 and ejection head 70 illustrating the flow of fluid 74 from the fluid cartridge 72 to a cell ejection chamber 78 and out of a cell ejection port 80. In FIG. 11, the fluid 74 containing cells 76 is provided through a supply via 82 etched through a semiconductor substrate 84 through fluid supply channels 86 in an aspiration channel layer 88 to the cell ejection chambers 78. Each of the cell ejection chambers 78 is in fluid flow communication with an aspiration channel 90 that is connected by a common aspiration channel 92 to an aspiration port 94. In some embodiments, shown in FIG. 12, an aspiration via 96 is also etched through the semiconductor substrate 84. FIG. 12 also shows a flow 98 of the fluid 74 containing cells 76 from a fluid reservoir in the cartridge body 100 through the supply via 82 to the cell ejection chamber 78 wherein a portion of the fluid is drawn off through the aspiration channel 90 in the direction of arrow 102 and a single cell 76 and a minor amount of fluid 104 are heated by the thermal cell ejector 18 (not shown) to cause the minor amount of fluid 104 and cell 76 to be expelled through the cell ejection port 80. The aspiration pressure is selected to be sufficient to draw off some of the fluid 74 in the cell ejection chamber 78 so that only a minor amount of fluid remains with the cell 76 in the cell ejection chamber 78. The minor amount of fluid 104 is an amount of fluid sufficient to be heated by the thermal cell ejector 18 so that the minor amount of fluid 104 and cell 76 can be expelled through the cell ejection port 80. In the embodiment illustrated in FIGS. 11 and 12, there is no need to invert the fluid cartridge 72 before use since the fluid 74 containing cells 76 is fed directly to the cell ejection chamber 78 from a supply of fluid 74 in the cartridge body 100. As with all of the embodiments described herein, the aspiration channel 90 is large enough to draw off fluid from the fluid in the cell ejection chamber, yet small enough to prevent a cell 76 from traveling through the aspiration channel 90. Accordingly, the cell 76 operates similar to a valve to plug the aspiration channel when a cell is situated in the cell ejection chamber 78 as shown in FIG. 12.

It will be appreciated that the foregoing embodiments provide a single cell dispensing device that is compact, inexpensive, and can accurately place one cell at a time in a desired location on an analytical substrate with the precision of conventional fluid ejection technology.

It is noted that, as used in this specification and the appended claims, the singular forms “a,” “an,” and “the,” include plural referents unless expressly and unequivocally limited to one referent. As used herein, the term “include” and its grammatical variants are intended to be non-limiting, such that recitation of items in a list is not to the exclusion of other like items that can be substituted or added to the listed items

For the purposes of this specification and appended claims, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that can vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

While particular embodiments have been described, alternatives, modifications, variations, improvements, and substantial equivalents that are or can be presently unforeseen can arise to applicants or others skilled in the art. Accordingly, the appended claims as filed and as they can be amended are intended to embrace all such alternatives, modifications variations, improvements, and substantial equivalents.

What is claimed is:

1. A cell dispensing device comprising;
a thermal fluid ejection head comprising a plurality of cell ejection chambers formed in an aspiration channel layer attached to a first semiconductor substrate, a thermal cell ejector disposed on the first semiconductor substrate in each of the cell ejection chambers, and aspiration channels formed in the aspiration channel layer in flow communication with at least some of the cell ejection chambers;
a cell ejection nozzle layer containing cell ejection ports therein, wherein the cell ejection nozzle layer is attached to the aspiration channel layer;
an activation circuit for each thermal cell ejector in the plurality of cell ejection chambers; and
an aspiration device in fluid flow communication with at least some of the cell ejection chambers through the aspiration channels.

2. The cell dispensing device of claim 1, further comprising a barrier wall having a height ranging from about 50 microns to about 4 millimeters on the cell ejection nozzle layer circumscribing the cell ejection ports therein.

3. The cell dispensing device of claim 1, further comprising a fluid reservoir to which the thermal fluid ejection head is attached.

4. The cell dispensing device of claim 3, further comprising a fluid supply via through the first semiconductor substrate in fluid flow communication with the fluid reservoir, wherein the fluid supply via is configured for providing fluid through fluid supply channels to the cell ejection chambers.

5. The cell dispensing device of claim 3, wherein the thermal fluid ejection head further comprises a separate fluid ejection structure comprising a second semiconductor substrate having a plurality of fluid ejectors thereon and a fluid supply via etched therethrough, a flow feature layer attached to the second semiconductor substrate, and a nozzle plate attached to the flow feature layer, wherein the fluid supply via is in fluid flow communication with a fluid in the fluid reservoir, and wherein the separate fluid ejection structure is devoid of the aspiration channels in the flow feature layer.

6. The cell dispensing device of claim 5, further comprising a barrier wall having a height ranging from about 50 microns to about 4 millimeters on the cell ejection nozzle layer circumscribing the cell ejection ports therein.

7. The cell dispensing device of claim 1, wherein the cell ejection ports in the cell ejection nozzle layer have a diameter ranging from about 10 to about 50 microns.

8. The cell dispensing device of claim 1, wherein the cell ejection chambers have a width and length ranging from about 10 to about 60 microns and a depth ranging from about 15 to about 30 microns.

9. A method of capturing and dispensing individual cells to an analytical substrate comprising:
providing a cell dispensing device comprising:
a thermal fluid ejection head comprising a plurality of cell ejection chambers formed in an aspiration channel layer attached to a first semiconductor substrate, a thermal cell ejector disposed on the first semiconductor substrate in each of the cell ejection chambers, and aspiration channels formed in the aspiration channel layer in flow communication with at least some of the cell ejection chambers;
a cell ejection nozzle layer containing cell ejection ports therein, wherein the cell ejection nozzle layer is attached to the aspiration channel layer;
an activation circuit for each thermal cell ejector in the plurality of cell ejection chambers; and
an aspiration device in fluid flow communication with at least some of the cell ejection chambers through an aspiration port in fluid flow communication with the aspiration channels;
applying a fluid containing cells to the cell ejection nozzle layer;
applying a negative pressure to the aspiration port to pull cells in the fluid from the cell ejection nozzle layer through the cell ejection ports into the cell ejection chambers;
removing excess fluid containing cells from the cell ejection nozzle layer;
determining which cell ejection chambers have cells therein; and
activating the thermal cell ejectors for the cell ejection chambers having cells therein to deposit the cells onto the analytical substrate.

10. The method of claim 9, wherein the cell ejection nozzle layer has a barrier wall having a height ranging from about 50 microns to about 4 millimeters circumscribing the cell ejection ports therein.

11. The method of claim 9, wherein the thermal fluid ejection head is attached to a fluid reservoir and the fluid reservoir comprises a separate fluid ejection structure comprising a second semiconductor substrate having a plurality of fluid ejectors thereon and a fluid supply via etched therethrough, a flow feature layer attached to the second semiconductor substrate, and a nozzle plate attached to the flow feature layer, wherein the fluid supply via is in fluid flow communication with a fluid in the fluid reservoir, and wherein the separate fluid ejection structure is devoid of the aspiration channels in the flow feature layer; further comprising activating the separate fluid ejection structure to deposit fluid onto the analytical substrate with the cells deposited onto the analytical substrate.

12. The method of claim 11, wherein the cell ejection nozzle layer has a barrier wall having a height ranging from about 50 microns to about 4 millimeters circumscribing the cell ejection ports therein.

13. The method of claim 9, wherein the cell ejection ports in the cell ejection nozzle layer have a diameter ranging from about 10 to about 50 microns.

14. The method of claim 9, wherein the cell ejection chambers have a width and length ranging from about 10 to about 60 microns and a depth ranging from about 15 to about 30 microns.

15. A method of capturing and dispensing individual cells to an analytical substrate comprising:
providing a cell dispensing device comprising:
a thermal fluid ejection head attached to a fluid reservoir, the thermal fluid ejection head comprising a plurality of cell ejection chambers formed in an aspiration channel layer attached to a semiconductor substrate, a thermal cell ejector disposed on the semiconductor substrate in each of the cell ejection chambers, and aspiration channels formed in the aspiration channel layer in flow communication with at least some of the cell ejection chambers;
a cell ejection nozzle layer containing cell ejection ports therein, wherein the cell ejection nozzle layer is attached to the aspiration channel layer;
an activation circuit for each cell ejector in the plurality of cell ejection chambers; and
an aspiration device in fluid flow communication with at least some of the cell ejection chambers through an aspiration port in fluid flow communication with the aspiration channels;
applying a negative pressure to the aspiration port to pull a fluid containing cells from the fluid reservoir into the cell ejection chambers;
removing excess fluid containing cells from the cell ejection chambers;
determining which cell ejection chambers have cells therein; and
activating the thermal cell ejectors for the cell ejection chambers having cells therein to deposit the cells onto the analytical substrate.

16. The method of claim 15 wherein the semiconductor substrate comprises a fluid supply via through the semiconductor substrate in fluid flow communication with the fluid reservoir and the cell ejection chambers.

17. The method of claim 15, wherein the cell ejection ports in the cell ejection nozzle layer have a diameter ranging from about 10 to about 50 microns.

18. The method of claim 15, wherein the cell ejection chambers have a width and length ranging from about 10 to about 60 microns and a depth ranging from about 15 to about 30 microns.
